(19) United States
(12) Patent Application Publication
(10) Pub. No.: US 2024/0108805 A1
(43) Pub. Date: Apr. 4, 2024

(54) SYSTEM AND METHOD FOR PROVIDING AN ADJUSTABLE FLOW RATE

(71) Applicant: PAVmed, Inc, New York, NY (US)

(72) Inventors: Lishan Aklog, New York, NY (US); Richard Yazbeck, New York, NY (US); Michael Boutillette, New York, NY (US); Jessie Gifford, New York, NY (US); Peter Aliski, New York, NY (US); Amos Cruz, New York, NY (US); Jonathan O'Keefe, New York, NY (US)

(21) Appl. No.: 18/275,601

(22) PCT Filed: Feb. 2, 2022
(86) PCT No.: PCT/US2022/014834

§ 371 Date: Aug. 2, 2023

Related U.S. Application Data
(60) Provisional application No. 63/144,829, filed on Feb. 2, 2021.

(51) Int. Cl. A61M 5/168

(52) U.S. Cl. CPC A61M 5/16804; A61M 5/16877; A61M 2205/3334

(57) ABSTRACT

Methods and apparatuses for providing an adjustable flow resistor. The adjustable flow resistor can include a chamber into which fluid is introduced, having an input end and an output end. A flow modifier can be situated within the chamber, between the input end and the output end, to modify a flow rate of the fluid. An adjustable resistance member can be positioned to interact with the flow modifier within the chamber to controllably modify the constant flow rate out of the adjustable flow resistor.

### CROSS-REFERENCE TO RELATED APPLICATION

This application claims priority to, and the benefit of, U.S. Provisional Application No. 63/144,829, filed Feb. 2, 2021, for all subject matter common to both applications. The disclosure of said provisional application is hereby incorporated by reference in its entirety.

### FIELD OF THE INVENTION

The present disclosure relates to an adjustable variable flow resistor suitable for adjusting and maintaining a target flow rate. In particular, the present disclosure relates to a flow resistor that can provide a constant flow rate and is adjustable or tunable to change and establish a new and different constant flow rate.

### BACKGROUND

Many of fluid transfer applications require that the fluid flow is controlled to deliver a substance to a location at a specified rate. Flow can be controlled by setting the pressure differential, the resistance, or both. These can be actively controlled but such systems require active pressure sources (e.g., pumps) or resistors (e.g., valves) often with feedback loops based on flow sensors.

Controlling flow completely passively, however, is more difficult. Passive flow resistors (e.g., manual or fixed valves, orifice plates, etc.) are commonly used to control flow but their accuracy are dependent on maintaining a fairly constant pressure. This is typically accomplished with a large reservoir of fluid, (relative to the volume of fluid to be delivered) with stored potential energy that is constant (e.g., elevated tank). A major limitation of this passive variable resistor design is that it is structurally linked to the infusion device and its design is dependent on the device. Perhaps more importantly, its specifications are dependent on the initial conditions, specifically the initial pressure, and the specific trajectory of the pressure for that specific device. The functionality of passive variable resistors would be greatly enhanced and available to a broader set of applications if its design and structure were independent of the pressure source and fluid reservoir and that its resistance was simply a function of the instantaneous pressure difference P at least over a specified range.

One example of a fluid transfer application is patient infusions. Infusions remain ubiquitous in healthcare spanning a wide range of conditions, substances, access sites and venues. Despite advances in oral and other drug delivery modes (e.g., transdermal, inhaled) many critical therapies still require intravenous (IV) infusion. It is estimated that one million infusions are administered per day in the United States. Over 90% of hospitalized patients receive an IV infusion. Infused substances can include drugs (e.g., antibiotics, chemotherapy, pain medications, local anesthetics, vasoactive agents, biologics), fluids (e.g., crystalloids, colloids, parenteral nutrition), and blood products (e.g., red cells, plasma, platelets). These substances are typically infused as (1) a single bolus volume (a few ml to several liters) over a limited time period (e.g., minutes to hours) or (2) a continuous infusion delivered a fixed or titrated rate (typical range 0.1 ml to 5 ml per minute).

Infusions can be administered through a variety of routes, most commonly intravenous but also intraarterial, subcutaneous, intrapleural, intraarticular, epidural and intrathecal, intraperitoneal, and intramuscular. A wide variety of catheters are available to facilitate infusions in through these various routes. Although traditionally, infusions have been administered in hospital settings, an increasing number of patients are receiving infusions in ambulatory infusion centers and at home. Because these latter settings have fewer, less skilled clinical personnel, only certain infusions are deemed to be safe there such as intravenous antibiotics, certain chemotherapeutic agents, local anesthetics for postoperative pain control, and certain narcotic pain medications.

Healthcare infusions are generally driven by relatively stale technologies such as gravity, active displacement electric pumps, or non-electric disposable elastomeric pumps. All three have well known disadvantages. Gravity driven infusions have low capital and disposable costs but require careful monitoring by a nurse, are not very accurate, limit patient mobility, and have no patient safety features. Electric pumps are accurate (±3%), have built in safety features of debatable efficacy but are expensive, bulky, susceptible to human factors, and limit mobility. Additionally, electronic infusion pump errors are a serious ongoing problem and represent a large share of the overall human and economic burden of medical errors. Electronic infusion pumps have become expensive and high maintenance devices, which have been plagued in recent years by recalls due to serious software and hardware problems. These pumps are designed for fine adjustments of infusions in complex patients, such as those in a critical care setting, and their use for routine infusions is technologic overkill. In terms of outpatient infusions, disposable pumps are convenient and fairly inexpensive but have no patient safety features and can be highly inaccurate (±15-40%) and are therefore unsuitable for use with medications where flow accuracy is critical, such as chemotherapeutic. The FDA's MAUDE database includes numerous reports of complications and even deaths resulting from disposable infusion pump flow inaccuracies.

The landmark 1999 Institute of Medicine report, “To Err is Human” (REF), attributed 40-100,000 deaths per year in the U.S. to medical errors. Medication errors, 40% of which are serious, life-threatening, or fatal, are the most common medical error and cost the health care system billions of dollars per year. Intravenous medication errors are the most common medication error and over 35% of these are related to infusion pumps. Studies have shown that despite progressively feature-laden “smart pumps”, human factors, software and hardware issue continue to contribute to serious errors (REF). The FDA's MAUDE Adverse Event reporting system contain numerous examples of serious injury and death related to infusion pump errors, both electric and disposable.

Thus, there is a need in the industry for effective, safe, passive fluid transfer devices, such as for example, for healthcare infusions. There is a need for improvements for modifying, adjusting, and providing a consistent flow rate with such devices.

### SUMMARY

In accordance with example embodiments of the present invention, an adjustable flow resistor is provided. The adjustable flow resistor includes a housing having an inlet to receive a fluid; a flow chamber disposed in the housing and having a flow modifier disposed within the flow chamber to provide a reduced cross-sectional flow path defined by a flow channel between the flow modifier and an inner surface of the flow chamber, the flow modifier being movable within the flow chamber to vary a length of the flow channel; and a resistance member disposed within the housing to apply a force on the flow modifier. The resistance member having adjustable properties to set the force being applied to the flow modifier and affect the length of the flow channel so as to maintain a constant flow rate of the fluid moving through the flow chamber.

In accordance with aspects of the present invention, the adjustable flow resistor can further include an adjustment mechanism coupled to the resistance member to modify an attribute of the resistance member to controllably modify the constant flow rate. The adjustment mechanism can include a lumen extending along a length of the adjustment mechanism such that the fluid exits the adjustable flow resistor through a distal end of the adjustment mechanism, and the fluid flows through the lumen at the constant flow rate.

In accordance with aspects of the present invention, the resistance member can be a spring or a coil. The adjustment mechanism can include a thread on an outer surface thereof and the thread can receive at least a portion of the spring or the coil to prevent a received portion of the spring or the coil from compressing. The adjustment mechanism can be rotatable to adjust a length of the spring or the coil received by the thread.

In some embodiments, the adjustable flow resistor can include a stopper situated between the flow chamber and a distal end of the adjustment mechanism. The resistance member can be a pressurized gas chamber. The resistance member can be a spring, and adjustments to the resistance member include adjusting a biasing constant of the spring.

In accordance with example embodiments of the present invention, an adjustable flow system is provided. The adjustable flow system includes a first pathway for directing fluid from a fluid reservoir; an adjustable flow resistor having its input end in communication with the first pathway for receiving a flow of fluid from the fluid reservoir, the adjustable flow resistor including: a flow chamber having a flow modifier disposed within the flow chamber to provide a reduced cross-sectional flow path defined by a flow channel between the flow modifier and an inner surface of the flow chamber, the flow modifier being movable within the flow chamber to vary a length of the flow channel; a resistance member disposed within the adjustable flow resistor to apply a force on the flow modifier, the resistance member having adjustable properties to set the force being applied to the flow modifier and affect the length of the flow channel so as to maintain a constant flow rate of the fluid moving through the flow chamber; and an adjustment mechanism coupled to the resistance member, the adjustment mechanism being configured to modify the adjustable properties of the resistance member; and a second pathway in communication with an output end of the adjustable flow resistor to direct fluid exiting the adjustable flow resistor.

In accordance with aspects of the present invention, the resistance member can be a spring or coil. The adjustment mechanism can constrain at least a portion of the spring or coil. The adjustment mechanism can be rotatable to adjust a length of the spring or coil constrained. The resistance member can be, alternatively, a pressurized gas chamber. The adjustable flow system can further include a stopper situated between the flow chamber and a distal end of the adjustment mechanism. The adjustable properties can include a biasing constant. The adjustment mechanism can include a lumen extending along a length of the adjustment mechanism to an outlet and the fluid can flow through the lumen at the constant flow rate.

In accordance with example embodiments of the present invention, a method for adjusting a flow rate is provided. The method includes introducing a flow of fluid into a chamber having a flow modifier moveably situated within the chamber and having a flow channel defined by a gap between the flow modifier and an inner surface of the chamber; applying a first force from the fluid flow against a proximal end of the flow modifier to affect a length of the flow channel within the chamber to passively provide a constant rate of fluid flow through the chamber; setting a biasing constant of a resistance member to apply a second force against a distal end of the flow modifier to affect movement of the flow modifier within the chamber and modify the constant rate of fluid flow through the chamber.

In accordance with aspects of the present invention, the method can further include outputting the fluid at a predetermined constant flow rate that is independent of the flow rate of the introduced flow of the fluid into the inlet of the chamber. The setting step can include rotating an adjustment mechanism to adjust a compressible length of the resistance member to set the biasing constant of the resistance member.

### BRIEF DESCRIPTION OF THE FIGURES

These and other characteristics of the present disclosure will be more fully understood by reference to the following detailed description in conjunction with the attached drawings, in which:

FIG. 1 is a perspective view of an example embodiment of the adjustable flow resistor, in accordance with the present disclosure;

FIGS. 2A and 2B are side views of an example embodiment of the adjustable flow resistor with a transparent housing, in accordance with the present disclosure;

FIGS. 3A and 3B are cross-sectional side view of an example embodiment of the adjustable flow resistor with a transparent housing, in accordance with the present disclosure; and

FIG. 4 is a chart showing example adjustable flow rates for different infusion parameters, in accordance with the present disclosure.

### DETAILED DESCRIPTION

An illustrative embodiment of the present disclosure relates to a flow resistor that can be adjusted or tuned to provide different consistent/continuous flows regardless of the input flow rate. The flow resistor of the present disclosure can be configured to be adjustable to select a desired constant flow rate, then provide that desired constant flow rate, and can subsequently be modified to provide a different constant flow rate at any given time within a predefined range of flow rates.

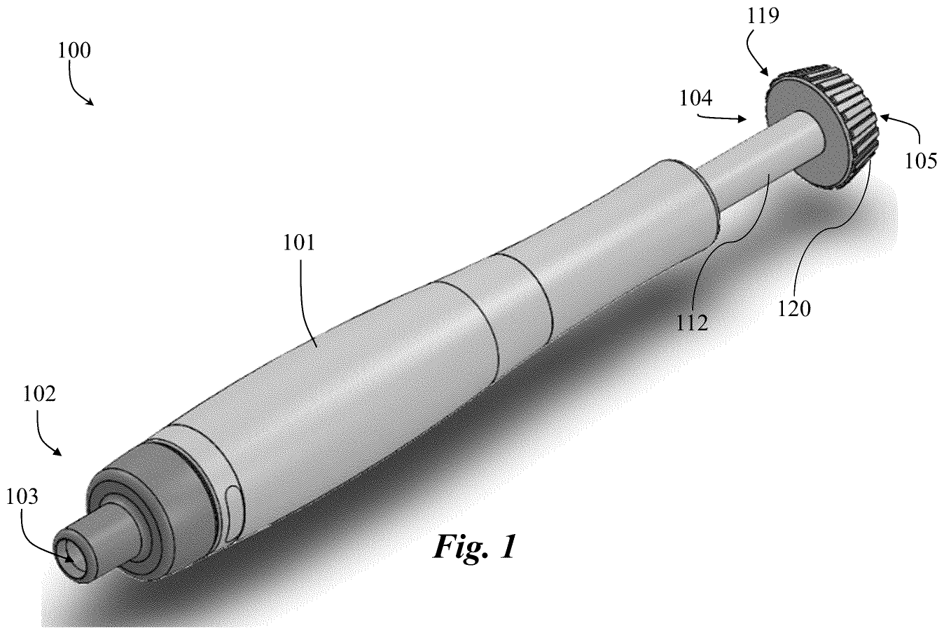

FIGS. 1 through 4, wherein like parts are designated by like reference numerals throughout, illustrate an example embodiment or embodiments of improved operation for an adjustable flow resistor, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIG. 1, an adjustable flow resistor 100 is provided. The adjustable flow resistor 100, in an embodiment, can have a proximal end 102 with an inlet 103 for receiving a fluid flow directed from, for instance, a fluid reservoir, such as an IV bag, via one or more pathways, e.g., plastic tubing. The adjustable flow resistor 100 can also have an outlet 105 at distal end 104 through which fluid can exit the adjustable flow resistor 100. As seen in the FIG. 1, the adjustable flow resistor 100 can be provided with housing 101 extending between the proximal end 102 and the distal end 104, and within which housing 101 various components can be provided to modify fluid flow entering inlet 103 to a desired fixed flow rate before being directed through the outlet 105, as will be discussed herein. Outlet 105, in one embodiment, can be designed to accommodate tubing to direct fluid flow to a desired destination or site of interest, e.g., an intravenous line in a patient.

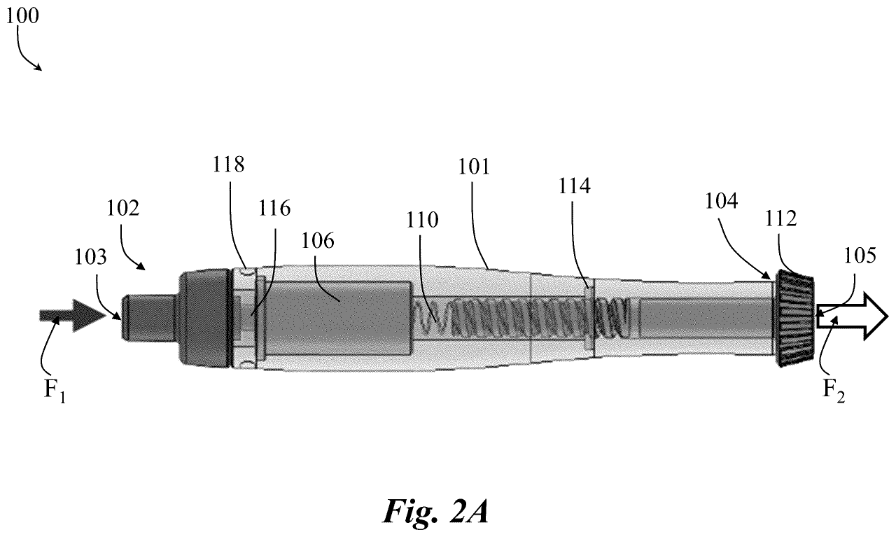

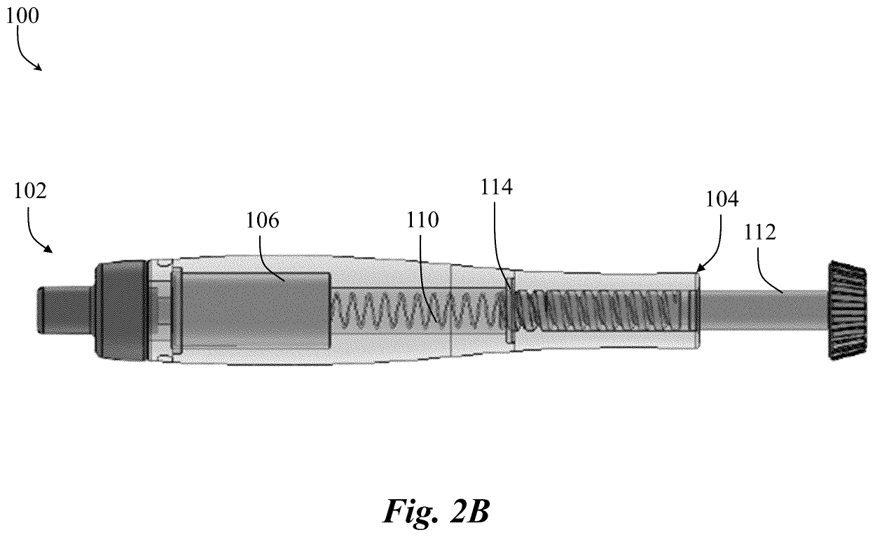

The adjustable flow resistor 100, in accordance with an embodiment of the present invention, can be used as part of a fluid flow system in which it is desirable to have a consistent (i.e., constant, or fixed) flow rate. The adjustable flow resistor 100 can provide selectable constant flow rates, within a pre-defined range, to provide flexibility when different constant flow rates are desired. The adjustable flow resistor 100, in one embodiment, can be designed to receive an input flow F₁, via the inlet 103, then generate an output flow F₂ at a desired constant flow rate through the outlet 105, regardless of the flow rate of input flow F₁ received at the inlet 103 or any backflow pressure at the outlet 105, as shown in FIG. 2A. In other words, the adjustable flow resistor 100 can receive an input flow F₁ at a first rate then modify that fluid flow, as discussed in greater detail herein, to a desired output flow F₂ having a constant flow rate. The inlet 103 can be provided with any size and shape to complementarily receive fluid flow from a source (not shown) through tubing (not shown). In one embodiment, an adapter (not shown) can also be provided to permit connection between the inlet and the fluid source (e.g., with a catheter or tube). Similarly, the outlet 105 can be provided with any size and shape to complimentarily receive a tube to deliver the fluid to a desired location (e.g., a patient IV line).

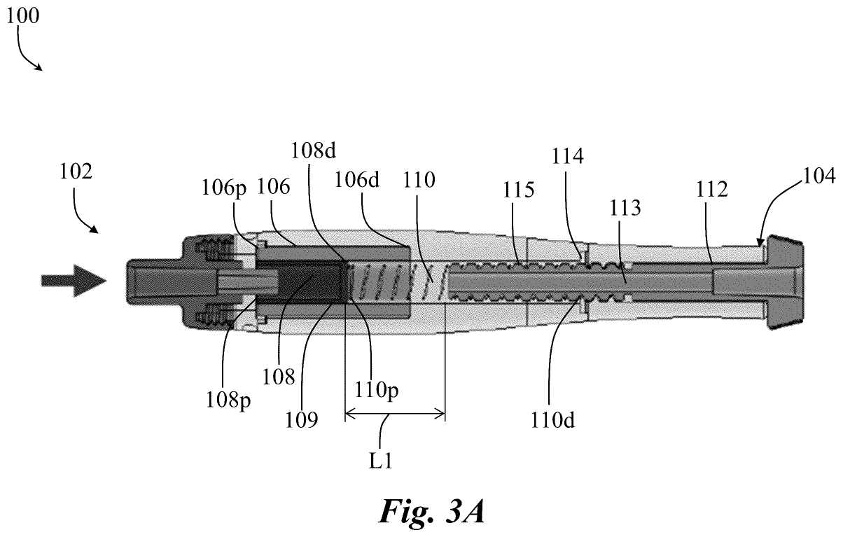

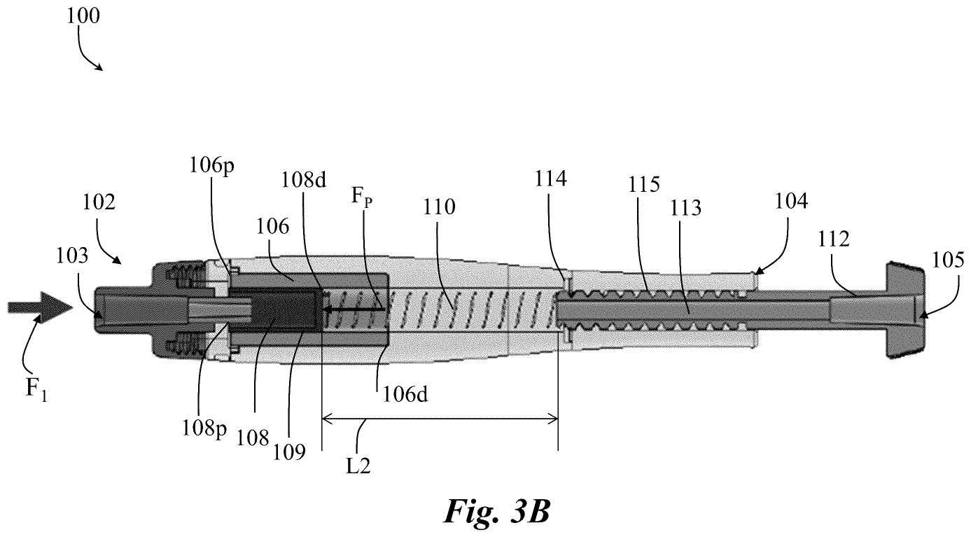

Looking now at FIGS. 2A, 2B, 3A, and 3B, in some embodiments, the adjustable flow resistor 100 can include a flow chamber 106 (or cylinder) for receiving the input flow F₁ entering through the inlet 103 at an input flow rate. As illustrated, the flow chamber 106 can be arranged within the housing 101 at a location distal to the inlet 103. The flow chamber 106, in one embodiment, can be provided with any size or shape so long as the flow chamber 106 is capable of receiving a fluid flow directed through the inlet 103 at the proximal end 102 of the adjustable flow resistor 100. In the illustrated embodiment, the flow chamber 106 can have a generally cylindrical shape. The adjustable flow resistor 100 can also include a flow modifier 108 (or piston), as illustrated in FIGS. 3A and 3B, situated, at least partially, between a proximal end 106p and a distal end 106d of a flow chamber 106 to allow for passive adjustments to the flow rate of the input flow F₁. The relative position of the flow modifier 108 within the flow chamber 106 provides a gap between an outer surface of flow modifier 108 and an inner surface of the flow chamber 106 to define a flow channel 109. In that way, when the flow modifier 108 moves distally further into the flow chamber 106, the length of flow channel 109 increases, and when the flow modifier 108 moves proximally out of the flow chamber 106, the length of the flow channel 109 decreases. It should be appreciated that flow modifier 108 can be imparted with any geometric cross-sectional shape so long as it can be received within flow chamber 106. In one embodiment, both the flow chamber 106 and the flow modifier 108 can be cylindrical in shape. In addition to adjusting the rate of fluid flow through the chamber 106 by increasing or decreasing the length of the flow channel 109, the flow modifier 108, in accordance with an embodiment of the present invention, can modify or adjust a flow rate of fluid moving through the flow chamber 106 by modifying other properties (e.g., width, length, shape, volume, etc.) of a flow channel 109 within the flow chamber 106. It should be appreciated that the flow modifier 108 can also include any combination of mechanisms designed to modify the amount and/or rate of fluid that can flow through the flow channel 109 within the flow chamber 106, such that the fluid exits the outlet 105 of the adjustable flow resistor 100 at a desired predetermined constant, or fixed, flow rate.

As shown in FIGS. 3A and 3B, the flow modifier 108 can include a proximal end 108p and a distal end 108d. In an embodiment, the distal end 108d of the flow modifier 108 can engage a resistance member 110, such as a spring, to resist movement of the flow modifier 108 in a distal direction in response to a force or pressure being applied by input flow F₁ against the proximal end 108p of the flow modifier 108, as input flow F₁ moves through inlet 103. The balance of pressures, or forces, applied to the proximal end 108p and the distal end 108d of the flow modifier 108 can determine the position of the flow modifier 108 within the flow chamber 106 to increase or decrease the length of the flow channel 109 within the flow chamber 106 to modify the flow rate of the input flow F₁ and impart a substantially constant flow rate to the output flow F₂ as it exits the flow chamber 106. The flow modifier 108 in combination with the flow chamber 106 can be similar to the moveable elements/pistons discussed within U.S. patent application Ser. No. 16/845,752, hereby incorporated by reference herein in its entirety.

It should be appreciated that the resistance member 110 can be provided with linear elastic properties (e.g., it obeys Hooke's Law such as conventional springs, elastomeric bands, etc.), to provide a custom and predefined relationship between the pressure at the inlet 103 and the pressure at the outlet 105, such that the output flow F₂ is one of a constant, or consistent, flow rate that is independent of any pressure differential between the inlet 103 and outlet 105. The linear elastic properties can be defined by a biasing constant, e.g., a spring constant, for resistance members which have elastic properties. As an example, the adjustable flow resistor 100 may have its inlet 103 be in fluid communication with a fluid reservoir, e.g., IV bag, and its outlet 105 be in fluid communication with a vein of a patient. In such a setup, proximal end 108p of the flow modifier 108 can be exposed to fluid pressure from input flow F₁ as it enters through inlet 103, while the distal end 108d of the flow modifier 108 can be exposed to a venous pressure from a patient's vein through outlet 105. The distal end 108d of the flow modifier 108 can further be exposed to a force Fp from resistance member 110, as seen in FIG. 3B. The balance of the forces acting on the distal end 108d and that act on proximal end 108p (i.e., pressure differential) determines the movement and location of the flow modifier 108 into and within the flow chamber 106.

It should further be appreciated that movement of the flow modifier 108 into the flow chamber 106 can create a reduced cross sectional flow channel 109, or reduced cross-sectional flow path, with the flow chamber 106, thus increasing resistance to fluid flow across the flow chamber 106. This reduced cross-sectional flow channel 109 can be further influenced by the location of the flow modifier 108 within the flow chamber 106. In particular, as flow modifier 108 moves distally and further into the flow chamber 106, length of flow channel 109 can increase, thereby increasing the distance and resistance to the fluid flow across the flow chamber 106. Specifically, by directing the input flow F₁ through the flow channel 109, the resulting fluid flow can be slowed due to the laminar flow of the fluid through a relatively narrow flow channel 109. When the flow channel 109 is longer, the input flow F₁ can be slowed further relative to when the flow channel 109 is shorter. Thus, the adjustable flow resistor 100, as provided herein, can modify and control fluid flow from a reservoir as it moves through the flow channel 109 to allow the output flow F₂ to exit outlet 105 at a substantially consistent, or constant flow rate regardless of the pressure differential acting on the flow modifier 108 or changes to the input pressure and/or an input flow F₁. In that way, the adjustable flow resistor 100 can prevent or minimize complications associated with fluid infusion that may proceed too fast or too slow.

It should be noted that the adjustable flow resistor 100, as disclosed herein, can be incorporated into any combination of systems that require a consistent flow rate of fluid from a fluid source to a site of interest. In one example, the adjustable flow resistor 100 can be implemented within an intravenous infusion set and disposable infusion pumps for routine inpatient and outpatients infusions respectively. Implementation into infusion sets will permit hospitals to return to gravity-based infusions and eliminate expensive electric infusion pumps for most inpatient infusions. The accuracy of the variable flow resistor incorporated into a disposable infusion pump can also allow outpatient administration of a broader range of drugs, thereby significantly expanding the addressable market.

Referring again to FIGS. 2A and 2B, in some embodiments, the resistance member 110 can include any combination of components, or mechanisms, so long as resistance member 110 can provide a force that can influence or modify movement of the flow modifier 108 within the flow chamber 106. For example, the resistance member 110 can be one, or a combination, of a spring, coil, a pressurized gas chamber, a gas piston chamber, a flexible member, a series of stoppers/dividers, etc. acting on the distal end 108d of the flow modifier 108 and/or the flow chamber 106 itself. The force FP being applied by the resistance member 110 to the flow modifier 108, as noted herein, can be an opposing force to that being applied to proximal end 108p of the flow modifier 108. In that way, the balance of these forces can affect the flow rate of fluid moving through the adjustable flow resistor 100.

In some embodiments, the biasing constant of resistance member 110 can be variably set, for example, with the adjustment mechanism 112 to establish the force FP being applied to the distal end 108d of flow modifier 108 in order to yield different constant flow rates. For example, moving adjustment mechanism 112 proximally towards inlet 103 effectively results in a shortened resistance member 110 (i.e., spring or coil) having a first compressible length L1, as seen in FIG. 2A and FIG. 3A. It should be noted that resistance member 110 is effectively shortened but not compressed to set the biasing constant of the resistance member 110. The biasing constant of the resistance member can be a function of the compressible length L1, L2 of the resistance member 110. Therefore, a shortened resistance member 110, as it can be appreciated, may apply a greater magnitude of force FP to counter the force from the fluid pressure of the input flow F₁, thus increasing the constant flow rate of the output flow F₂ through the outlet 105, due to the established biasing constant of the resistance member 110. Thus, the greater magnitude of force applied by the first compressible length L1 of resistance member 110 to the distal end 108d of the flow modifier 108 can minimize movement of the flow modifier 108 distally within the flow chamber 106 and can thereby create a shorter flow channel 109. This shorter flow channel 109 can result in relatively less resistance to the input flow F₁ leading to a relatively faster constant flow rate through the chamber 106.

In contrast to the shortened resistance member 110 of FIG. 2A and FIG. 3A, a longer resistance member 110, resulting when adjustment mechanism 112 is moved distally away from inlet 103, can be provided with a second compressible length L2 that is longer than the first compressible length L1, as seen in FIG. 2B and FIG. 3B, to set the biasing constant of the resistance member 110. Imparting resistance member 110 with compressible length L2 can result in an application of a lesser or lower magnitude of force FP to counter the forces from the fluid pressure of input flow F₁, thus decreasing the flow rate of the output flow F₂ through the outlet 105, as a result of the variably set biasing constant. Specifically, with a lower magnitude of force FP being applied by the second compressible length L2 of the resistance member 110 to the distal end 108d of the flow modifier 108, the flow modifier 108 can move further distally within the flow chamber 106 than in the case of a shorter resistant member 110, resulting in a relatively longer flow channel 109. This longer flow channel 109 can result in additional resistance to the input flow F₁ leading to a relatively slower constant flow rate through the chamber 106.

In embodiments where the resistance member 110 is a spring, the adjustment mechanism 112 can be coupled to the resistance member 110 and can be designed to modify a compressible length L1, L2 of at least a portion of the resistance member 110. The compressible length L1, L2 of the resistance member 110 can be modified by the adjustment mechanism 112 using any combination of mechanisms. In some embodiments, to modify the compressible length of the resistance member 110, the adjustment mechanism 112 can include threads 115, or grooves, designed to complement the shape/pitch of the resistance member 110. The distal end 119 of the adjustment mechanism 112 can include a textured knob portion 120 that can provide a user with tactile feedback or added grip when the user is actuating the adjustment mechanism 112. As the adjustment mechanism 112 is threaded into the resistance member 110, to accommodate the resistance member circumferentially within/about the threads 115, those portions of the resistance member 110 surrounding the adjustment mechanism 112 can be constrained to prevent that portion from being compressed. By positioning the resistance member 110 within (or withdrawing from) the threads 115 of the adjustment mechanism 112 the compressible length L1, L2 of the resistance member 110 can be modified to thus change the biasing constant of the resistance member 110. The greater the portion of the resistance member 110 within the threads 115 of the adjustment mechanism 112 the lesser of the portion of the resistance member 110 acting on the flow modifier 108. Adjusting the length of the resistance member 110 does not necessarily involve compressing the resistance member 110 but adjusting the compressible length, L1, L2 of the resistance member 110 which acts on the flow modifier 108. In alternative embodiments, other adjustment mechanisms 112 are contemplated to constrain the compressive, or active, length of the resistance member 110. For example, in some embodiments, the adjustment mechanism 112 can include multiple adjustment points along a length of the adjustable flow resistor 100. In such cases, the multiple adjustment points can be buttons, sliders, etc. that, once activated, will adjust the resistance member 110 to the setting associated with those adjustment points. For instance, adjustment mechanism 112 can be a series of buttons disposed along a side of housing 101. The buttons can be slidably received within through holes that extend perpendicular to a central axis of the adjustable flow resistor such that the buttons can be depressed into the housing. The buttons can be depressed inward, into the housing, to be situated between the space of adjacent coils of resistance member 110. As any one of the buttons is depressed into the resistance member 110, the compressible length, e.g., L1, L2, of the resistance member 110 can be changed to effect movement of the flow modifier 108, as described in detail above. The placement of the buttons in series along the side of housing 101 can represent predefined flow rates.

In some embodiments, the adjustment mechanism 112 can be manipulated to adjust a compressive length, e.g., L1, L2, of the resistance member 110 applying a force to the flow modifier 108. For example, the adjustment mechanism 112 can be rotatable, pushable, pullable, etc. to adjust a length of the resistance member 110 (e.g., how much of the resistance member 110 is meshed with the adjustment mechanism 112). In the illustrated embodiment, the adjustment mechanism is a rotatable pin with at least one thread 115 extending outward from an outer surface thereof. The threads 115 are able to interact with the helical turns of the resistance member 110 to constrain a portion of the resistance member 110.

In some embodiments, the adjustable flow resistor 100 can include one or more stoppers 114 situated between the flow chamber 106 and a distal end of the adjustment mechanism 112 to assist in modifying the resistance member 110. The stopper 114 can be provided to segment the resistance member 110 and to adjust a constant flow rate for the adjustable flow resistor 100. The stopper 114 can provide a surface for the resistance member 110 to sit on and allow the adjustment mechanism 112 to be able to move back and forth because the stopper 114 can engage the threads in adjustment mechanism 112. In some embodiments, the adjustable flow resistor 100 can include multiple removable stoppers 114 each at different locations along a length of the housing 101 to change the effective length of the resistance member 110. An example of a stopper or multiple stoppers, in one embodiment, can be a button or series of buttons (not shown) situated along housing 101 which can be pushed in between coils of the resistance member 110 to change the effective length of the resistance member 110.

In some embodiments, as seen in FIGS. 3A and 3B, the adjustment mechanism 112 can include a hollow channel, or lumen, 113 to provide a flow path to permit fluid to flow from the flow chamber 106 through the channel 113 within or around the resistance member 110, into and through or around the adjustment mechanism 112, and out the outlet 105 at the distal end 104 of the adjustable flow resistor 100.

Continuing with FIGS. 3A and 3B, the elements of the adjustable flow resistor 100 in combination can provide a continuous flow path from the inlet 103 to the flow chamber 106 through the flow channel 109 through adjustment mechanism 112 and to the outlet 105. For example, a fluid flow can enter through inlet 103 of the adjustable flow resistor 100 and be fed into the flow chamber 106. The flow chamber 106 and the flow modifier 108 can modify the input flow rate received at the chamber 106 to be output by the adjustable flow resistor 100. As discussed herein, the constant rate of the flow will depend on the amount of force being applied by the resistance member 110, which can be adjusted using the adjustment mechanism 112 to different constant flow rates. For example, the adjustment mechanism 112 can be rotated to capture the resistance member 110 within threads 115 to change the compressible length of the resistance member 110. Thereafter, the fluid flow can exit the flow chamber 106, pass through and/or around the resistance member 110 and into a channel within the adjustment mechanism 112. The adjustment mechanism 112 can have an outlet at its distal end for outputting the fluid at the desired flow rate.

In some embodiments, the adjustable flow resistor 100 can be implemented as part of a system for delivering a consistent fluid flow. The system can include one or more tubes for transporting a fluid flow and introduce the flow into the adjustable flow resistor 100. The flow chamber 106 can be provided to receive the fluid flow from the one or more tubes and an outlet from the adjustment mechanism 112 for outputting the fluid at the flow rate to the one or more tubes.

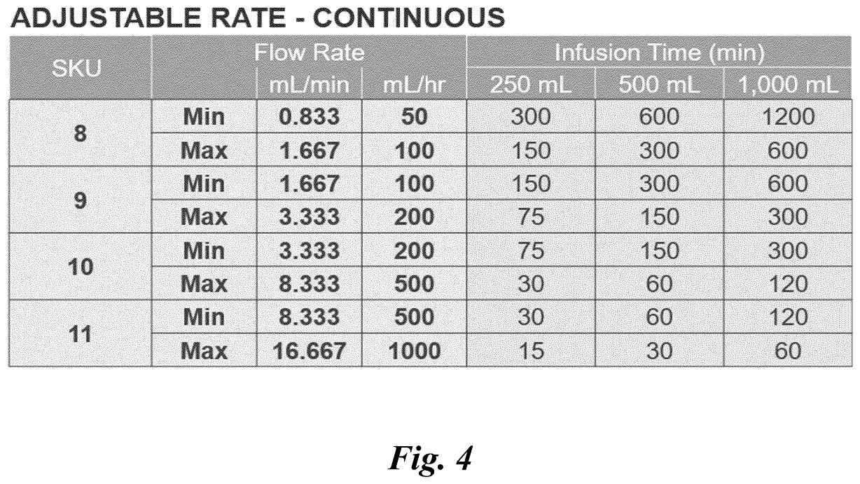

In use, the adjustment mechanism 112 can modify the constant flow rate through the flow resistor 100 from a predefined minimum constant flow rate to a predefined maximum constant flow rate, as seen in FIG. 4. For example, in a first embodiment, the minimum constant flow rate may be 50 ml/hr and the maximum constant flow rate can be 100 ml/hr. The adjustment mechanism 112 can be used to set the biasing constant of the resistance member 110 by changing the effective compressible length L1, L2 of the resistance member 110. In the case where the resistance member 110 is a spring, the biasing constant can be the spring constant k. By setting the biasing constant of the resistance member 110, the resistance member 110 can affect the movement of the flow modifier 108 within the flow chamber 106 to result in a new constant flow rate through the adjustable flow resistor 100. Once the constant flow rate is established the infusion time for a known volume of fluid can be easily determined. In one example, if the adjustment mechanism 112 is set such that the flow modifier has a constant output flow F₂ of 50 ml/hr with a 250 ml bag of fluid, it can be expected that the fluid infusion time will be approximately 200 minutes. As seen in FIG. 4, additional ranges of flow rates and infusion times are contemplated, including those not explicitly noted in FIG. 4. In some embodiments the adjustment mechanism can include markers, or indicators, which represent the desired output flow F₂.

In some embodiments, the adjustable flow resistor 100 can include one or more indicators 116 designating when a flow is active, the status of the flow, the flow rate, etc. The one or more indicators 116 can include any combination of visible, audio, tactile, etc. indicators conveying a status of the flow through the adjustable flow resistor 100. For a visual indicator 116, the body of the adjustable flow resistor 100 can include a transparent window 118 showing at least a position of a portion of the mechanical components inside the adjustable flow resistor 100, as seen in FIG. 2A. The mechanical components, for example the flow modifier 108, can provide a visual cue(s) to the user showing when a flow is active. For example, when a flow is not active, the flow modifier 108 can rest near a proximal end of the adjustable flow resistor 100 such that it covers an indicator 116 portion located under the window 118. When a flow is active, the flow modifier 108 can be moved in the proximal direction exposing the indicator 116 such that a user can see the indicator 116 (e.g., a green piece) under window 118 conveying an active flow. This simple implementation permits the patient, or medical professional, to know whether the adjustable flow resistor 100 is functioning as intended.

In operation, the adjustable flow resistor 100 can be disposed between a fluid source and a desired output to provide a consistent flow rate which can be adjusted to provide other consistent flow rates. For example, depending on the relationship between the resistance member 110 and the adjustment mechanism 112 the consistent, or fixed, flow rate will be set to a second consistent, or fixed, flow rate. A method for adjusting the flow rate can include providing, or introducing, a fluid flow to an adjustable flow resistor at an initial input flow F₁. The adjustable flow resistor 100 can include a flow chamber 106 for receiving the fluid flow F₁ and a flow modifier 108 which together can define a flow channel 109 to control the flow rate through the flow chamber 106. The adjustable flow resistor 100 can additionally include a resistance member 110 in contact with the flow modifier 108 and an adjustment mechanism 112 for modifying an attribute of the resistance member 110. The method can also include setting a desired constant output flow F₂ on the adjustment mechanism 112 to modify the attribute of the resistance member 110 to modify the input flow F₁ to the output flow F₂. The adjustment mechanism 112 can adjust the output flow rate within predefined minimum and maximum flow rates, as seen in FIG. 4. In some embodiments, the method can include rotating the adjustment mechanism to modify the attribute of the resistance member.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. An adjustable flow resistor comprising:
a housing having an inlet to receive a fluid of an input flow;
a flow chamber disposed in the housing;
a flow modifier disposed within the flow chamber, the flow modifier configured to define a flow channel between the flow modifier and an inner surface of the flow chamber, the flow channel having one or more flow channel property, the flow modifier configured to move within the flow chamber and modify a flow channel property in response to a pressure of the input flow acting on the flow modifier;
a resistance member disposed within the housing and configured to apply a force on the flow modifier, the resistance member having one or more resistance member property configured to affect movement of the flow modifier; and
an adjustment mechanism configured to adjust a resistance member property and controllably modify a constant flow rate of the fluid moving out of the flow chamber.

2. The adjustable flow resistor of claim 1, wherein the resistance member property the adjustment mechanism is configured to adjust comprises an elastic property.

3. The adjustable flow resistor of claim 1,
wherein the adjustment mechanism includes a lumen extending along a length of the adjustment mechanism such that the fluid exits the adjustable flow resistor through a distal end of the adjustment mechanism, and
wherein the fluid flows through the lumen at the constant flow rate.

4. The adjustable flow resistor of claim 1, wherein the adjustment mechanism comprises a plurality of adjustment points, when activated an adjustment point is configured to modify the constant flow rate to a predefined flow rate associated with the adjustment point.

5. The adjustable flow resistor of claim 1, wherein:
the resistance member is a spring or a coil; and
the adjustment mechanism is configured to constrain at least a portion of the spring or the coil.

6. The adjustable flow resistor of claim 5, wherein the adjustment mechanism is rotatable and configured to adjust a length of the spring or the coil constrained by the adjustment mechanism.

7. The adjustable flow resistor of claim 1, further comprising a stopper situated between the flow chamber and a distal end of the adjustment mechanism.

8. The adjustable flow resistor of claim 1, wherein the resistance member is a pressurized gas chamber.

9. The adjustable flow resistor of claim 1,
wherein the resistance member property the adjustment mechanism is configured to adjust comprises at least one of a biasing constant, a compressible dimension of the resistance member, and an active dimension of the resistance member.

10. An adjustable flow system comprising:
a first pathway for directing fluid from a fluid reservoir;
an adjustable flow resistor having its input end in communication with the first pathway for receiving a flow of fluid from the fluid reservoir, the adjustable flow resistor including:
a flow chamber;
a flow modifier disposed within the flow chamber, the flow modifier configured to define a flow channel between the flow modifier and an inner surface of the flow chamber, the flow channel having one or more flow channel property, the flow modifier configured to move within the flow chamber and modify a flow channel property in response to a pressure of the flow of fluid acting on the flow modifier;
a resistance member disposed within the adjustable flow resistor and configured to apply a force on the flow modifier, the resistance member having one or more resistance member property configured to affect movement of the flow modifier; and
an adjustment mechanism configured to adjust a resistance member property and controllably modify a constant flow rate of the fluid flow exiting the adjustable flow resister; and
a second pathway in communication with an output end of the adjustable flow resistor to direct fluid exiting the adjustable flow resistor.

11. The system of claim 10, wherein the resistance member property the adjustment mechanism is configured to adjust comprises an elastic property.

12. The system of claim 10, wherein:
the resistance member is a spring or coil; and
the adjustment mechanism is configured to constrain at least a portion of the spring or coil.

13. The system of claim 12, wherein the adjustment mechanism is rotatable to adjust a length of the spring or coil constrained by the adjustment mechanism.

14. The system of claim 10, wherein the resistance member is a pressurized gas chamber.

15. The system of claim 10, further comprising a stopper situated between the flow chamber and a distal end of the adjustment mechanism.

16. The system of claim 10, wherein the resistance member property the adjustment mechanism is configured to adjust comprises at least one of a biasing constant, a compressible dimension of the resistance member, and an active dimension of the resistance member.

17. The system of claim 10,
wherein the adjustment mechanism includes a lumen extending along a length of the adjustment mechanism to an outlet, and
wherein the fluid flows through the lumen at the constant flow rate.

18. A method for adjusting a flow rate, the method comprising:
introducing a flow of fluid into a chamber having a flow modifier moveably situated within the chamber and having a flow channel defined by a gap between the flow modifier and an inner surface of the chamber;
applying a first force from the fluid flow against a proximal end of the flow modifier to affect a length of the flow channel within the chamber to passively provide a constant rate of fluid flow through the chamber;
setting an elastic property of a resistance member to apply a second force against a distal end of the flow modifier to affect movement of the flow modifier within the chamber and modify the constant rate of fluid flow through the chamber.

19. The method of claim 18, further comprising outputting the fluid at a predetermined constant flow rate that is independent of a flow rate of the introduced flow of fluid.

20. The method of claim 18, wherein the setting step comprises using an adjustment mechanism to adjust the elastic property of the resistance member.
